(19) United States
(12) Patent Application Publication
(10) Pub. No.: US 2024/0426494 A1
(43) Pub. Date: Dec. 26, 2024

(54) Indoor air quality control system

(71) Applicant: SANAS, INCORPORATED, Brundidge, AL (US)

(72) Inventor: Frank Ebert, Rüsselsheim (DE)

(73) Assignee: SANAS, INCORPORATED, Brundidge, AL (US)

(21) Appl. No.: 18/754,027

(22) Filed: Jun. 25, 2024

Related U.S. Application Data
(60) Provisional application No. 63/608,707, filed on Dec. 11, 2023.
(60) Provisional application No. 63/523,350, filed on Jun. 26, 2023.

(51) Int. Cl. F24F 8/24; A61L 9/14; F24F 11/72; F24F 110/50

(52) U.S. Cl. CPC F24F 8/24; A61L 9/14; F24F 11/72; A61L 2209/111; F24F 2110/50

(57) ABSTRACT

A method for controlling an indoor climate (201, 202, 203) includes sensing air quality factors within a structure (101) using an air quality control system (100). The sensor data associated with the air quality factors is transmitted from the air quality control system (100) to a server (140). The server (140) analyzes the sensor data and calculates a target air quality value. The target air quality value is then transmitted from the server (140) to the air quality control system (100). The method further includes controlling disbursement of a biocidally active substance within the structure (101). The air quality factors include the sensed concentration of the biocidally active substance. The control of the disbursement of the biocidally active substance is performed using closed-loop control, with the target air quality value as a setpoint and the sensed concentration of the biocidally active substance as a process variable.

### TECHNICAL FIELD

The present disclosure relates to an air quality monitoring system, and more particularly the invention relates to a method for controlling an indoor climate, an indoor air quality control system, and a storage facility for perishable goods to control an indoor air quality.

### BACKGROUND

Indoor air quality is the air quality within and around buildings and structures. Indoor air quality is known to affect the health, comfort, and well-being of building occupants. A particular concern with respect to the health of occupants is airborne pathogens, in particular bacteria and viruses.

There is no generally accepted definition of the term “air quality,” and the term is used differently based on context. An air-conditioning technician may refer to air quality as a uniform air flow through an HVAC system due to regular filter changes and checks of the air ducts. Chip manufacturers or hospitals require particle and germ-free clean room air in a specific temperature range and humidity level in operating rooms.

In the field of indoor air supply, silo thinking tends to prevail among existing suppliers and products on the market. Some control the temperature, others the air flow rate, others specialize in filtering air, and still others in disinfecting air using UVC light, ionization, elaborate filter systems, or high-pressure spray systems. All of these systems and solutions lack an objective before and after measurement with all the necessary parameters of what air quality actually is and constitutes.

### SUMMARY

The present disclosure provides an indoor air quality control system that takes a comprehensive approach to sensing and controlling indoor air quality.

The system includes sensors to detect harmful particles and gases. Sensor data obtained from the sensors is analyzed, for example by comparing the sensor data with reference data. The analysis may be performed in “the cloud,” that is by computers arranged in computing centers away from the sensors. The analysis may include self-learning and optimizing strategies.

The system further includes actuators through which the indoor air quality in a room or in an entire building can be manipulated. In particular, the system may include a nebulizer for hydrogen peroxide (H₂O₂). The nebulizer may be controlled, based on the analyzed data from the sensors, to provide an optimal concentration of hydrogen peroxide in the air. An optimal concentration is the minimum concentration of hydrogen peroxide in the air that effectively destroys pathogens and minimizes the risk of transmitting airborne diseases. The optimal concentration is not a fixed value but depends on environmental factors obtained by the sensors. The environmental factors may include air temperature, humidity, and air pressure.

The system may be used to adjust further air quality parameters such as CO₂, O₂, Ozone, H₂S, H₂O₂, NH₃, ClO₂, and/or NO₂ particulate matter, humidity, temperature, and even air pressure to the reference concentrations and air quality, thus creating a desired, healthy room climate.

In another aspect, the system may control, either fully automatically or manually, the concentration of biocidally active substances in a room. The concentration of such biocidally active substances may be controlled with an accuracy of 0.1 ppm in the room air. Such precise concentration ensures the health and safety of occupants.

In particular, the system may include an optimized nebulizer for hydrogen peroxide. This optimized nebulizer utilizes one or more ultrasonic generators to produce fine particles from the liquid surface, which are then automatically dispersed into the air due to physical principles.

The system is particularly suitable for use in hospitals, government offices, schools, offices, shopping malls, high-rise buildings, theatres, cinemas, event venues, hotels, highly frequented places, but also long-vacant rooms. In a particularly beneficial application, the room may be the interior of an ambulance. The system helps to permanently improve the well-being and health of people and significantly reduce the risk of infection and thus the rate of disease and keep it stable in the long term.

In another application, the system may be used in a greenhouse to protect fruits and vegetables from diseases. The system may in particular be used to reduce exposure of fruits and vegetables to harmful bacteria and mold. The system may also be used in storage facilities for storing perishable items, including fruits and vegetables.

The following detailed description is merely exemplary in nature and is not intended to limit the invention or the application and uses of the invention. Furthermore, there is no intention to be bound by any theory presented in the preceding background or the following detailed description.

### BRIEF DESCRIPTION OF THE DRAWINGS

FIG. 1 shows an indoor air quality control system in a structure.

FIG. 2 shows indoor climate, or air quality, as a vector of different air quality factors.

FIG. 3 illustrates configuring of an air quality control system by a mobile phone.

FIG. 4 illustrates communication between an air quality control system and a cloud server.

FIG. 5 illustrates access to a cloud server from different locations.

FIG. 6A shows a top side of a printed circuit board, and FIG. 6B shows a bottom side thereof.

FIG. 7 shows a data visualization of air quality.

FIG. 8 shows an aspect of a user interface.

FIG. 9 shows a further aspect of a user interface.

FIG. 10 shows an overview page user interface.

FIG. 11 shows a header of a user interface page.

FIG. 12 shows a notification manager user interface.

FIG. 13 shows a sensors locations display user interface.

FIG. 14 shows a task view user interface.

FIG. 15 shows a daily report user interface.

FIG. 16 shows a sensor list user interface.

FIG. 17 shows a settings user interface.

FIG. 18 shows a live data visualization user interface.

FIG. 19 shows a live particle count data visualization user interface.

FIG. 20 shows an alternative live particle count data visualization user interface.

FIG. 21 shows a historical air quality data user interface.

### DETAILED DESCRIPTION

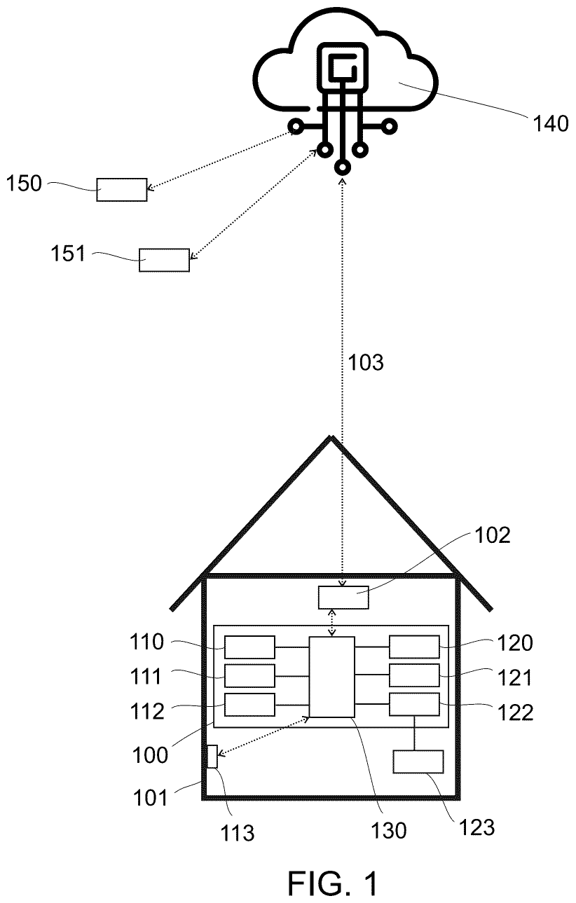

FIG. 1 shows an indoor air quality control system 100 that has been deployed in a structure 101. The structure 101 may be an entire building or a part of a building. The structure 101 may be a single room within a building. The structure 101 may be a hospital, an office building, a school, a shopping mall, a high-rise building, a theatre, a cinema, an event venue, a hotel, or another highly frequented place. The structure 101 may also be a vacant room, a greenhouse, or a storage facility, in particular a storage facility for perishable goods.

The system 100 includes a sensor 110 and an actuator 120. The system may include more than one sensor 110-112 and more than one actuator 120-123. The sensor 110 and the actuator 120 are operatively connected to a processor 130.

The processor 130 may be in communication with a cloud server 140. The cloud server 140 is typically arranged off-premises, that is outside the structure 101. The communication between the processor 130 and the cloud server 140 is facilitated by a router 102 that communicates with the cloud server 140 through a wide area network 103. The wide area network may be the Internet.

The processor 130 may be configured to buffer data obtained from the sensor 110 when the processor 130 is unable to communicate with the cloud server 140. Such buffered data may then be communicated to the cloud server 140 once communication between the processor 130 and the cloud server 140 has been re-established.

The processor 130, and thereby the indoor air quality control system 100, may communicate with the router 102 wirelessly, for example through a Wi-Fi connection.

The sensors 110-112 may be configured to sense one or more air quality factors. The air quality factors may include temperature, humidity, pressure, CO₂ concentration, O₂ concentration, ozone concentration, NA3, fine dust particle concentration, and spores concentration.

The indoor air quality control system 100 is configured to manipulate the air quality in the structure 101 by the actuator 120. The actuator 120 may be one of a plurality of actuators 120-123. The actuators 120-123 may include one or more actuators 123 that are part of the structure's 101 HVAC system. In particular, the actuator 122 may be a nebulizer, a blower, a dampener within an air duct, a humidifier, a dehumidifier, a heater, an air conditioner, a fragrance disburser, or a combination thereof.

The sensors 110-112, the actuators 120-123, and the processor 130 may be arranged within a common housing and operatively connected to one another by wires. Alternatively, one or more of the sensors 110-112 or one or more of the actuators 120-123 may be arranged in a separate housing, and wirelessly connected to the processor 130.

The indoor air quality control system 100 may be used to select a desired indoor climate from a list of preselected indoor climates. The term “indoor climate” here refers to at least two air quality factors that are jointly manipulated. For example, the indoor climate may be a two-dimensional vector that includes air temperature and humidity. The indoor climate may be a three-dimensional vector comprising temperature, humidity, and a concentration of hydrogen peroxide in the air. Generally, an “indoor climate” can be represented by an n-dimensional vector, wherein each dimension of the vector represents one air quality factor.

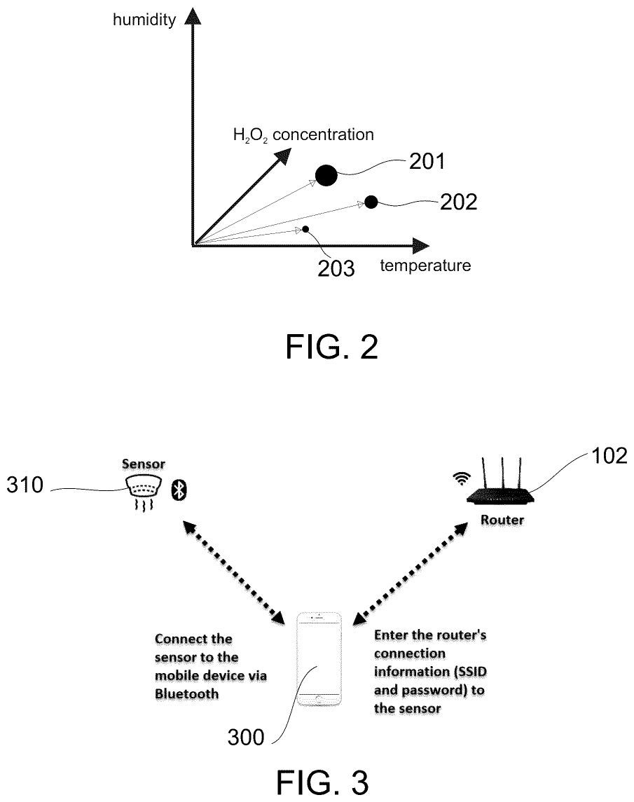

As illustrated in FIG. 2, the indoor air quality control system 100 may be configured to maintain a “coastal” indoor climate 201, a “desert” indoor climate 202, or a “mountain” indoor climate 203. The “coastal” indoor climate 201 may be characterized by a lower temperature, lower air pressure, higher humidity, and higher H₂O₂ concentration in the air than the “desert” indoor climate 202. By selecting an indoor climate, a user does not select any one air quality factor in isolation but rather selects two or more air quality factors combined.

In combination with perishable goods storage, the indoor climate may be optimized for the type of goods stored. For example, the indoor air quality control system 100 may be configured to maintain an “apples storage,” “potatoes storage,” “citrus storage,” and so forth optimized climate. The indoor climate for storage of perishable goods may in particular include an oxygen concentration and hydrogen peroxide concentration as air quality factors.

The indoor climate preferably includes at least one air quality factor that reflects a concentration of a biocidally active substance. In FIG. 2, the biocidally active substance is hydrogen peroxide. By selecting an indoor climate, the user automatically selects a target hydrogen peroxide concentration in the structure 101. Within a preselected indoor climate, the target hydrogen peroxide concentration is optimized to match the minimal concentration at which hydrogen peroxide is biocidally active to destroy airborne pathogens and thereby effectively prevent infection at the target humidity and target temperature.

A target hydrogen peroxide concentration for residential buildings is in the range of 0.05-0.3 ppm. A target hydrogen peroxide concentration for office buildings is in the range of 0.3-0.5 ppm. A target hydrogen peroxide concentration for high-frequented retail buildings and other public structures is in the range of 0.5-1 ppm. A target hydrogen peroxide concentration for medical buildings and other health-sensitive structures depends on the usage of this room and is in the range of 1-10 ppm as an hourly maximum. These concentrations have been found effective against dangerous viruses, bacteria, resistant germs, and fungi.

In some applications, it may be beneficial to adjust the target hydrogen peroxide concentration based on an occupancy state of the structure within which the indoor air quality control system 100 is deployed. For example, within an office building the air quality control system 100 may maintain a first target hydrogen peroxide concentration during office hours, and a second target hydrogen peroxide concentration during non-office hours. Similarly, when deployed within an ambulance the air quality control system 100 may maintain a first target hydrogen peroxide concentration while the ambulance is in use, and a second target hydrogen peroxide concentration during non-use, in particular during a dedicated disinfection time following a use period. In such applications, the indoor air quality control system 100 may include an occupancy sensor.

The indoor air quality control system 100 addresses issues of mold control and permanent mold prevention within the structure 101. After mold spores have been detected within the structure 101 using test trays, the next step is to eliminate the mold quickly and effectively and to prevent further mold growth. Mold only forms under certain general conditions. Wall temperature, air temperature, and air humidity are the decisive parameters, which must be controlled in an automated and resource-optimized manner so that the dew point is never undershot. With the indoor air quality control system 100, these three parameters can be permanently monitored at critical points. For example, the indoor air quality control system 100 may include a battery-powered wall temperature sensor 113. The wall temperature sensor 113 is attached to an interior wall within the structure 101 and in wireless communication with the processor 130. A wall temperature sensed by the wall temperature sensor 113 is then one dimension of a “mold prevention” indoor climate. Within that climate, air humidity is set such that the dew point at which the air reaches 100% humidity is below the wall temperature. Thereby, mold can no longer develop within the structure 101. The mold that has already developed is eliminated due to the H₂O₂ nebulization. In particular, the “mold prevention” indoor climate may be characterized by a higher H₂O₂ concentration as compared to non-mold prevention climates. In particular, the “mold prevention climate” may be characterized by having a target H₂O₂ concentration that is between 4-5 times the target H₂O₂ concentration of a corresponding non-mold prevention climate. The “mold prevention climate” may have a target H₂O₂ concentration that is greater than 1 ppm.

For optimal mold control, more than one wall temperature sensor 113 may be used and wirelessly connected to the processor 130. In that case, the processor 130 may use the lowest wall temperature sensed by the plurality of wall temperature sensors 113 to control the temperature and humidity, and thus the dew point, in the structure 101.

An H₂O₂ nebulizer 121 may be one of the actuators 120-123 of the indoor air quality control system 100. The nebulizer 121 is configured to produce nebulized H₂O₂ particles that are so small that surfaces and electronic equipment in the structure 101 will not be damaged.

The indoor air quality control system 100 may utilize one or more remote sensors 150-151 to collect air quality reference metrics. The remote sensors 150-151 are in communication with the cloud server 140 through the wide area network 103. The remote sensors 150-151 are reference sensors that may be set up at selected locations around the globe. The locations may be selected for having known good air quality.

Air quality sensor data from the remote sensors 150-151 may then be used in the cloud server 140 to determine target air quality factors for the indoor air quality control system 100. That is, the indoor air quality control system 100 may be controlled by the cloud server 140 to mimic the air quality at a known location at which a remote sensor 150 has been set up. A home in Los Angeles that has been equipped with the indoor air quality control system 100 can mimic the air quality sensed by a remote sensor 150 set up in Germany's Black Forest. Odorants or smells of forest can be easily also nebulized into the room so that the simulated climate will be nearly 99% matching to the real climate of the selected place. Data received from one or more remote sensors 150-151 may be used to establish desirable ranges of air quality factors, for example by collecting and analyzing data from the one or more remote sensors 150-151 over the course of several months or even several years.

The hydrogen peroxide nebulizer 121 operates based on ultrasonic nebulization technology. The hydrogen peroxide nebulizer 121 is configured to generate hydrogen peroxide droplets having a diameter between 100 nm and 0.5 μm. This particle size range has been found beneficial in allowing particles to disburse and evenly spread in the air of a structure 101 while being fine enough to not cause any harm to surface within the structure 101.

Within the indoor air quality control system 100, the hydrogen peroxide nebulizer 121 is closed-loop controlled based on data obtained from a hydrogen peroxide sensor 111. That is, if the hydrogen peroxide concentration sensed by the hydrogen peroxide sensor 111 falls below a target value for a selected indoor climate, the processor 130 activates the hydrogen peroxide nebulizer 121. If the hydrogen peroxide concentration sensed by the hydrogen peroxide sensor 111 is above a target value for a selected indoor climate, the processor 130 deactivates the hydrogen peroxide nebulizer 121. Alternatively, the processor 130 may control a flow rate of hydrogen peroxide to the hydrogen peroxide nebulizer 121. The closed loop control maintains an actual hydrogen peroxide concentration in the structure 101 within ±0.1 ppm of a target value.

The indoor air quality control system 100 may be calibrated to include a volume of the structure 101 within which the indoor air quality control system 100 is deployed. The volume may be transmitted to and stored in the cloud server 140. The volume of the structure 101 may be used to optimize the closed loop control of the hydrogen peroxide nebulizer 121 in response to data obtained from the hydrogen peroxide sensor 111.

Closed-loop control of the hydrogen peroxide concentration within the structure 101 takes place in real time and as such is preferably executed in the processor 130. The closed loop control may be based on input received from the cloud server 140. The closed-loop control of the hydrogen peroxide concentration is maintained even if communication between the processor 130 and the cloud server 140 has been lost.

Additional air quality factors sensed by the sensors 110-112, including CO₂, O₂, Ozone, H₂S, H₂O₂, NH₃, ClO₂, and/or NO₂ particulate matter, humidity, temperature, and even air pressure other particles, will be used to control the hydrogen peroxide nebulizer 121. As soon as the CO₂ content in the air rises above 1000 ppm, additional H₂O₂ nebulization is started and further measures to improve the air are initiated via the HVAC interface which is one of the accessories available for this system.

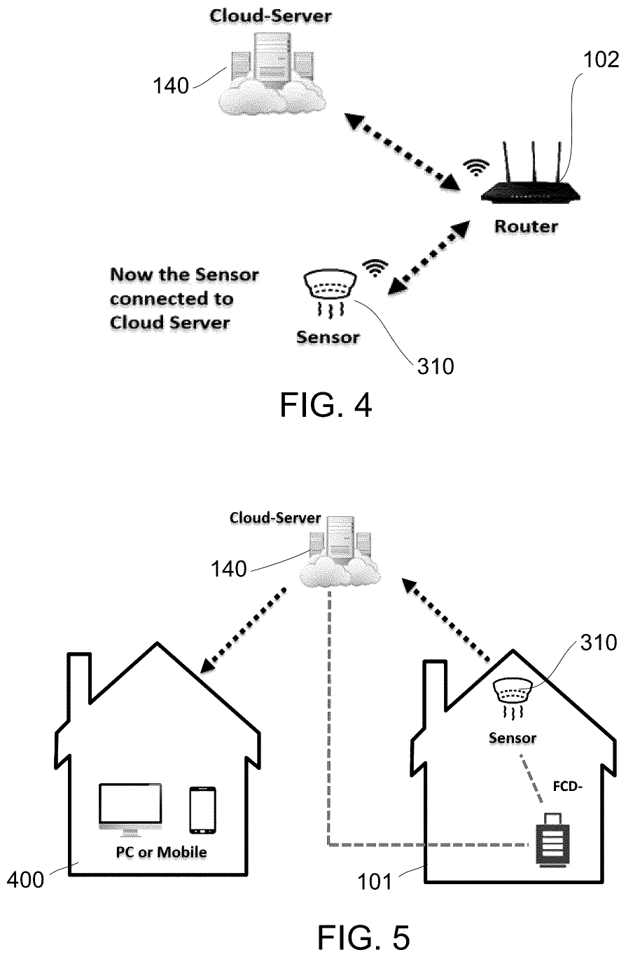

As illustrated in FIG. 1, the sensor 110 is in data communication with the cloud server 140. That is, the sensor 110 is operatively connected to the cloud server 140. Communication between the sensor 110 and the cloud server 140 may be established as shown in FIG. 3: In an initial state, the sensor 310 may be connected to a user interface device 300 such as a mobile phone. This initial connection may be a Bluetooth or WiFi connection. Through the initial connection, the sensor 310 may be configured to connect to a wireless router 102 present in the structure 101. As shown in FIG. 4, the sensor 310 may thereafter communicate with the cloud server 140 through the router 102.

The sensor 310 is shown in FIGS. 3 and 4 are to be understood to functionally refer to one or more of a plurality of sensors 110-113 that may be part of the indoor air quality control system 100. The sensor 310 is shown in FIGS. 3 and 4 also include the processor 130 to which electronic sensor elements within the sensors 110-113 are operatively connected.

The user interface device 300 may also be used to enter and associate a size of the structure 101 with the sensor 310 in the cloud server 140.

As shown in FIG. 5, data obtained from the sensor 310 in the structure 101 can be remotely obtained, through the cloud server 140, from anywhere in the world 400. Similarly, the indoor air quality control system 100 can be configured and a desired indoor climate can be selected from anywhere in the world 400 for the structure 101.

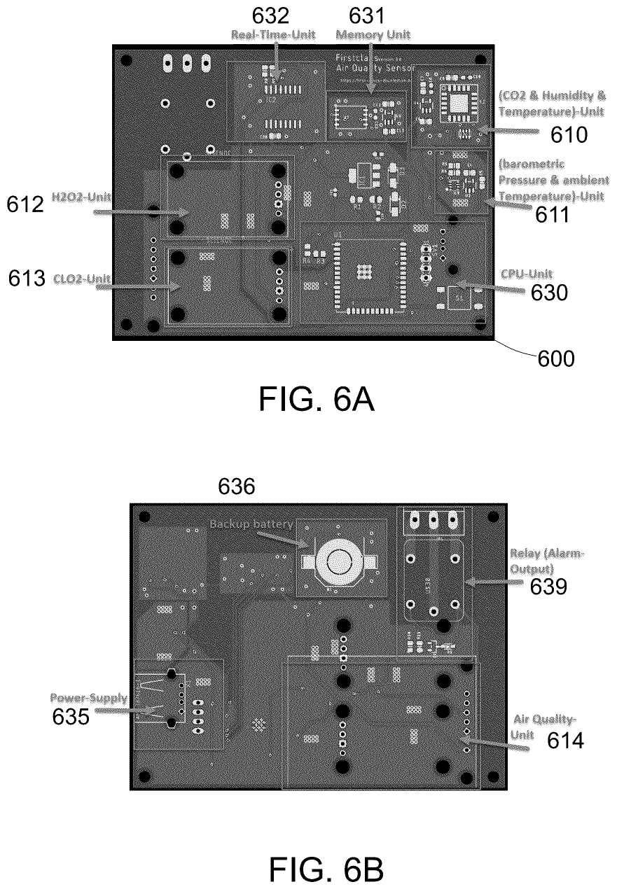

The indoor air quality control system 100 may utilize a printed circuit board 600 as shown in FIGS. 6A and 6B to accommodate several of the elements shown in FIG. 1. A top side of the printed circuit board 600 is shown in FIG. 6A. A bottom side of the printed circuit board, 600 is shown in FIG. 6B.

As shown in FIG. 6A, the printed circuit board 600 may include several sensor units. These include a CO₂, humidity, and temperature unit 610; a barometric pressure and ambient temperature unit 611; an H₂O₂ sensor unit 612, and a ClO₂ sensor unit 613. A further sensor in the form of an air quality unit 614 is arranged on the bottom side shown in FIG. 6B. One or more air quality factors may be redundantly measured by more than one sensor unit 610-614.

The CO₂, humidity, and temperature unit 610 can measure a CO₂ concentration within a measurement range of 400-5000 ppm with an accuracy of 40 ppm and a response time (r63%) of 60 s. It can measure a relative humidity within a measurement range of 0-95% RH and a response time (τ63%) of 120 s. It can measure temperature within a measurement range of −10 to 60° C. with an accuracy of 0.8° C. and a response time (τ63%) of 120 s.

The barometric pressure and ambient temperature unit 611 can measure barometric pressure with an RMS Noise of 0.2 Pa (equiv. to 1.7 cm), having a sensitivity Error of ±0.25% and a temperature coefficient offset ±1.5 Pa/K (equiv. to ±12.6 cm at 1° C. temperature change).

The H₂O₂ sensor unit 612 can detect hydrogen peroxide within a detection range of 0˜10 ppm. It has a response Time (T90)≤60 s and a repeatability ≤+2% signal. Its long-term output drift is <2% signal/month. Its operating temperature range is −20° C.˜40° C. Its operating humidity range is 15˜ 90% RH non-condensing. Its operating pressure range is 800˜ 1200 mbar.

The ClO₂ sensor unit 613 has a detection range of 0˜1 ppm. Its response time (T90) is ≤50 s. The ClO₂ sensor unit 613 has a repeatability <±2% signal and a long-term output drift <2% signal/month. Its resolution is <0.03 ppm. Its operating temp range is −20° C.˜ 40° C., its operating humidity range is 15˜ 90% RH non-condensing, and its operating pressure range is 800˜ 1200 mbar.

The air quality unit 614 can measure the presence of particles in a size ranges of 0.3˜1.0, 1.0˜2.5, and 2.5˜10 micrometers. It has a particle counting efficiency of 50% @ 0.3 m 98% @>=0.5 m. Its particle effective range (PM2.5 standard) is 0˜500 μg/m³. Its particle maximum range (PM2.5 standard) is ≥1000 μg/m³. The air quality unit 614 has a particle resolution of 1 μg/m³. The particle maximum consistency error based on the PM2.5 standard is ±10% @ 100-500 μg/m³(±10 μg/m 3 @0˜100 μg/m³). The particle standard volume is 0.1 liter. The single response time is <1 second and the total response time is ≤10 seconds. The air quality unit 614 has a working temperature range of −10˜ 60° C., and a working humidity range of 0˜99%.

The sensor units 610-614 are operatively connected to a CPU unit 630. The CPU unit includes a wireless interface with an output power of 20 dBm. The CPU unit 630 supports 802.11 b/g/n, Bluetooth, and BLE protocols. It operates in a frequency range between 2.4 GHz and 2.5 GHz.

The CPU unit 630 is connected to a memory unit 631 and a real-time unit 632. The memory unit 631 is a 4 Gbit (512 MByte) NOR Flash. The printed circuit board 600 is powered by a USB-type power supply 635 and backed by a type 1220 lithium metal button cell backup battery 636.

In the event of an internet outage or a problem with the router 102, the CPU unit 630 will save data obtained from the sensor units 610-614 inside the memory unit 631. When the internet connection is reestablished, the CPU unit 630 will send the saved data to the cloud server 140. The real-time unit 632 provides an accurate internal clock even in the event of an internet outage. The real-time unit 632 is backed by the backup battery 636 to maintain the date and time in case the printed circuit board 600 is disconnected from power.

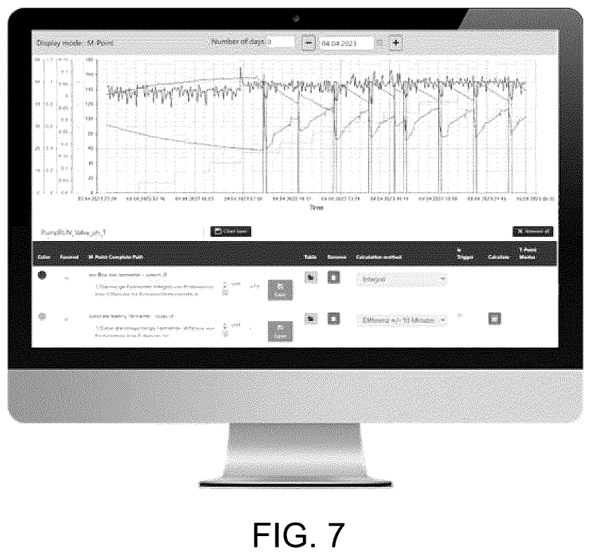

Data from the sensor units 610-614 is sampled at a rate of at least 1 sample per second. The data is then transmitted to the cloud server 140 where it is aggregated and stored long term. The data collected can be visualized and retrieved from the cloud server 140 in the form of graphs as shown in FIG. 7. The data stored in the cloud server 140 is also used to improve the functional performance of the indoor air quality control system 100. In case of air quality factors exceeding defined thresholds, the cloud server 140 can issue an alert to a user, for example in the form of a text message or push notification.

When an internet connection 103 is available, data collected from the sensors 110-113 is sent to the cloud server 140 in real-time. That is the indoor air quality control system 100 streams sensor data to the cloud server 140. The data stream originates from a single printed circuit board 600 which contains all of the necessary hardware in one compact form format. The data stream has an update rate of one or more data packets per second being sent from the indoor air quality control system 100 to the cloud server 140.

Indoor air quality, which may also be referred to as indoor climate, is a complex quantity that comprises several interrelated air quality factors. As such, it is difficult for a user to manually select any one air quality factor. The cloud server 140 aggregates and analyzes data from the indoor air quality control system 100 over time to establish the interrelation of air quality factors within the structure 101. The cloud server 140 provides a user interface that allows a user to select an indoor climate without having to worry about the various air quality factors that impact the indoor climate.

The user interface has been optimized for usability and includes several different user interface components.

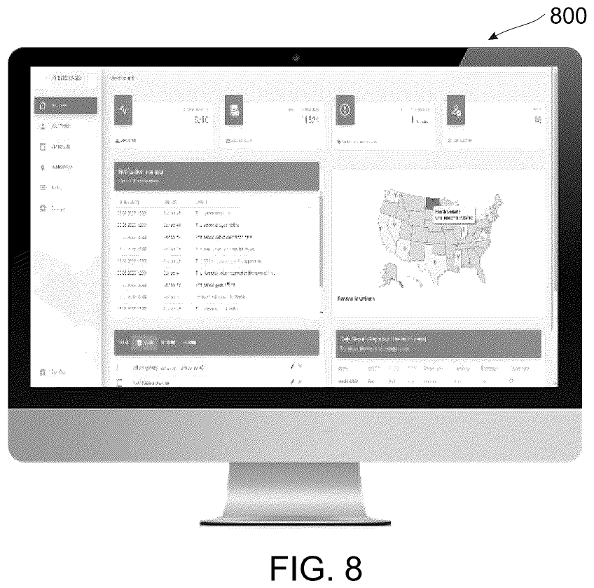

FIG. 8 shows a first user interface component 800. The user interface component 800 may be an HTML page served by the cloud server 140 or an app screen on a mobile device rendered in interaction with the cloud server 140. The first user interface component 800 is a dashboard to manage and monitor all sensors installed for a client. The cloud server 140 can support up to thousands of indoor air quality control systems 100, including managing operations, monitoring current data, setting the sensor, and downloading data.

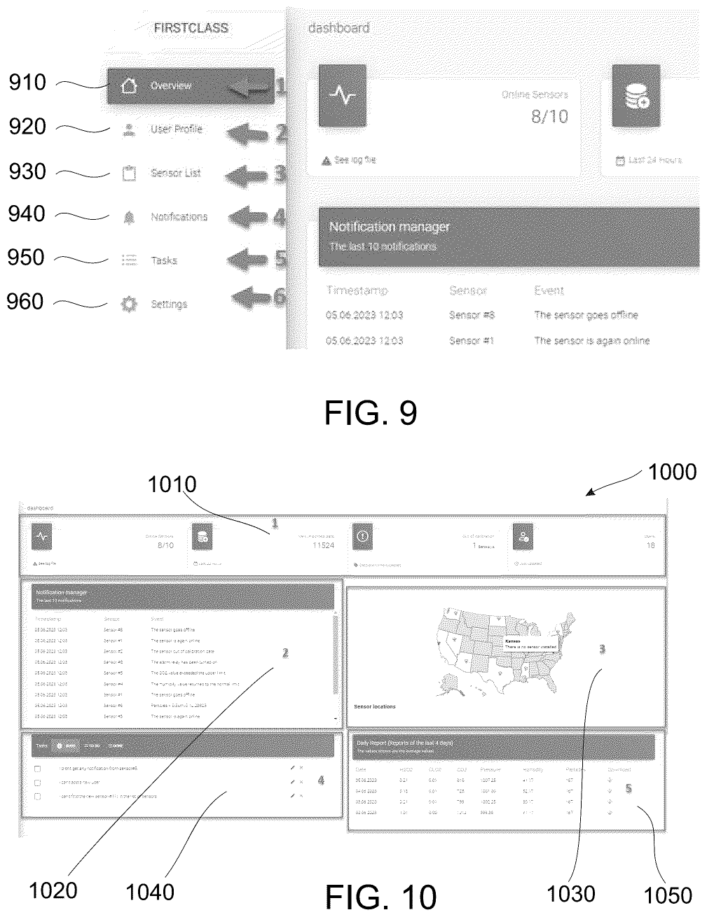

Referring now to FIG. 9, the user interface includes six main sections. An overview section 910 provides an overview of the current status of the cloud server 140, all the information and processes currently taking place in the cloud server 140, such as the currently active indoor air quality control systems 100, their last values, sensor locations, etc.

A user profile section 920 allows users to modify their account information and activate/deactivate notifications.

A sensor list section 930 displays a list of all indoor air quality control systems 100 associated with a user. The sensor list section 930 includes the last measured values and the last calibration date for each sensor. The sensor list section 930 provides access to modifying the settings of any sensor and following up the current values directly or viewing the previous values.

A notifications section 940 displays all notifications sent from the indoor air quality control systems 100. Notifications may indicate an indoor air quality control system 100 being offline or online again, measured values exceeding a certain threshold, or the measurement values being back under a threshold value.

A task section 950 allows users to indicate a problem that the user encountered in the cloud server 140 for resolution by the operator of the cloud server 140.

A settings section 960 allows manipulating general settings for the entire cloud server 140.

Referring now to FIG. 10, an overview page 1000 includes a header 1010, a notification manager 1020, a sensor locations display 1030, a task view 1040, and a daily report 1050.

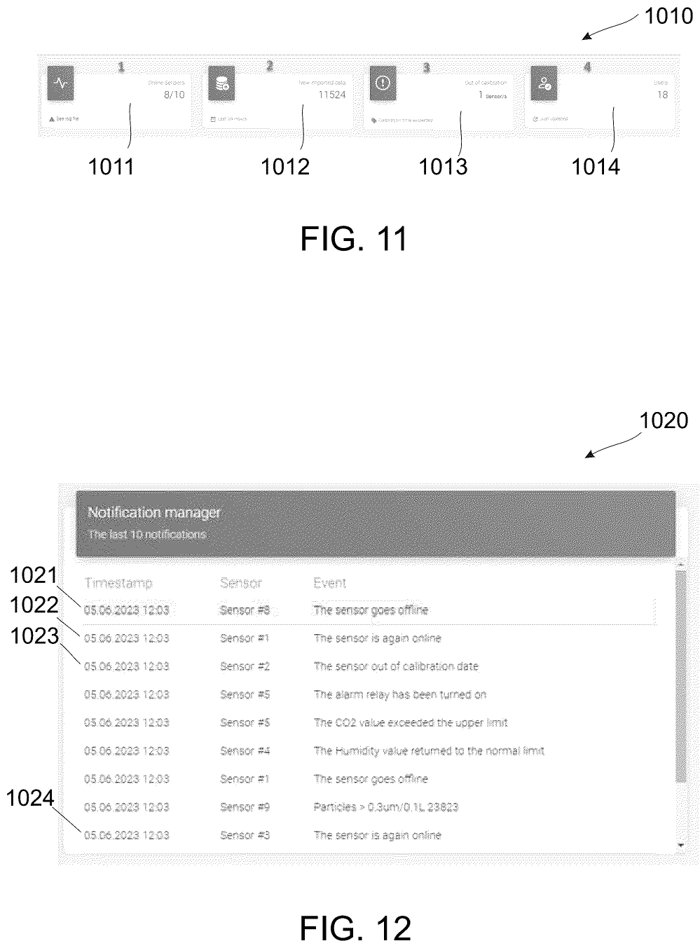

As shown in FIG. 11, the header 1010 includes four information cards. A first information card 1011 shows how many indoor air quality control systems 100 are online out of the total number of deployed indoor air quality control systems 100.

A second information card 1012 shows a number of raw data packets imported in the last 24 hours. One data packet may include a timestamp and associated sensor measurement values such as CO₂, O₂, Ozone, H₂S, H₂O₂, NH₃, ClO₂, NO₂ particulate matter, humidity, temperature, and even air pressure.

To deliver correct and reliable values, sensors 110-113 of an indoor air quality control system 100 must be periodically calibrated. A third information card 1013 shows how many indoor air quality control systems 100 include sensors that need to be calibrated.

A fourth information card 1014 shows a number of users that a logged into the cloud server 140.

FIG. 12 shows the notification manager 1020 in more detail. The notification manager 1020 displays a plurality of notifications 1021 . . . 1024. Each indoor air quality control system 100 sends notifications when a threshold value is exceeded or goes back below a threshold. The cloud server 140 also monitors the connected indoor air quality control systems 100. If an indoor air quality control system 100 stops sending data to the cloud server 140, the cloud server 140 creates a loss of communication notification 1021 that the indoor air quality control system 100 has been disconnected. After reconnecting the indoor air quality control system 100 to the cloud server 140, a reconnect notification 1022 is created. Notifications are displayed on the notification manager 1020 and may be transmitted to users in the form of text messages, push notifications, and the like. The notification manager 1020 shows the last ten notifications that have been created.

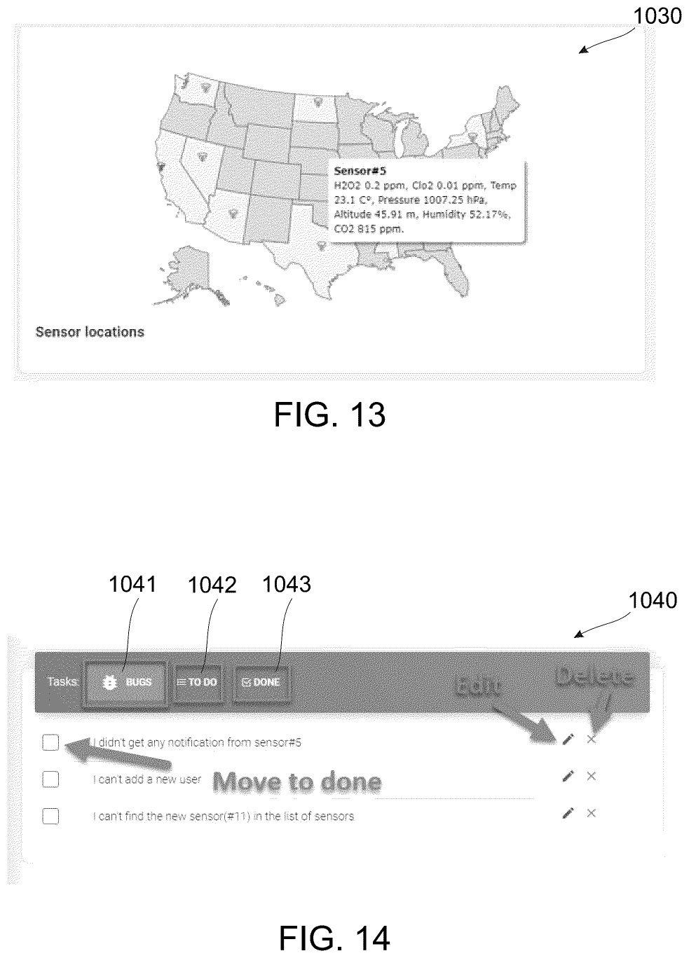

FIG. 13 shows the sensors locations display 1030 in detail. The sensors locations display 1030 provides a graphical user interface to illustrate a geographic location of the indoor air quality control systems 100. By hovering over a given location, last measured indoor air quality sensor values measured by a respective indoor air quality control system 100 are displayed.

FIG. 14 is a detailed illustration of the task view 1040. The task view 1040 provides a look at the last 10 tasks registered on the cloud server 140, which can be modified and deleted. The task view 1040 provides buttons to filter the task view. In particular, the task view can be filtered, by pressing a first button 1041, to display only tasks and technical problems addressed to a support team. The task view 1040 can be filtered, by pressing a second button 1042, to display unfinished tasks. The task view 1040 can be filtered, by pressing a third button 1043, to display finished tasks.

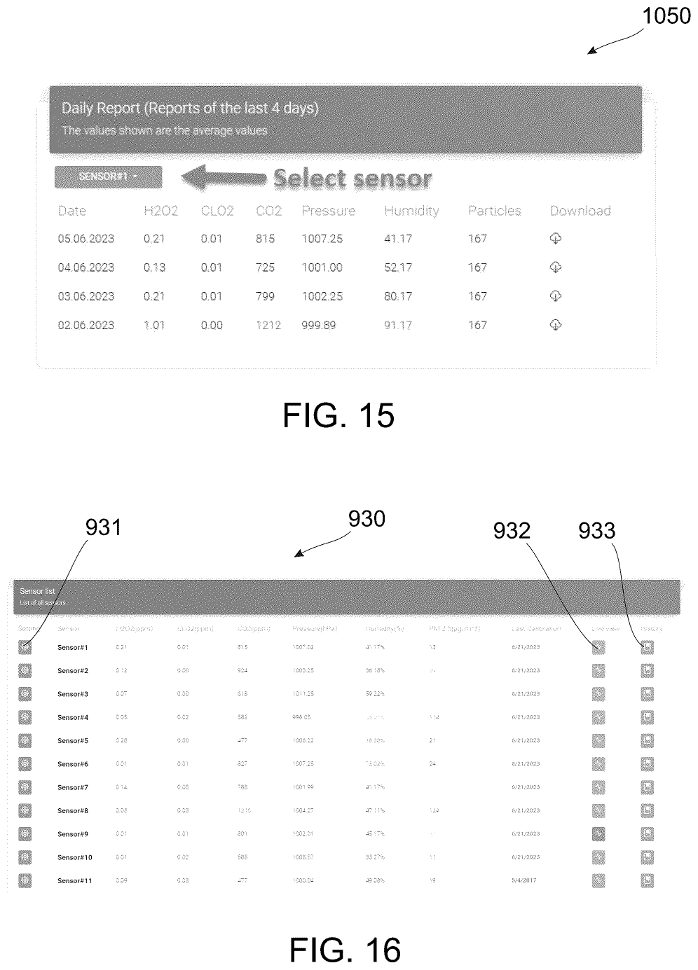

FIG. 15 is a detailed illustration of the daily report 1050. The daily report 1050 shows measured mean values for the last four days, which a user can view or download as an excel file.

FIG. 16 is a detailed illustration of the sensor list section 930. The sensor list section 930 shows all indoor air quality control systems 100 registered in the server 140 with their last sensor values. A user can modify the settings of any indoor air quality control system 100, by pressing an associated settings button 931. A user can view the live sensor values by pressing an associated live view button 932. The user can alternatively view historical sensor values by pressing an associated history button 933.

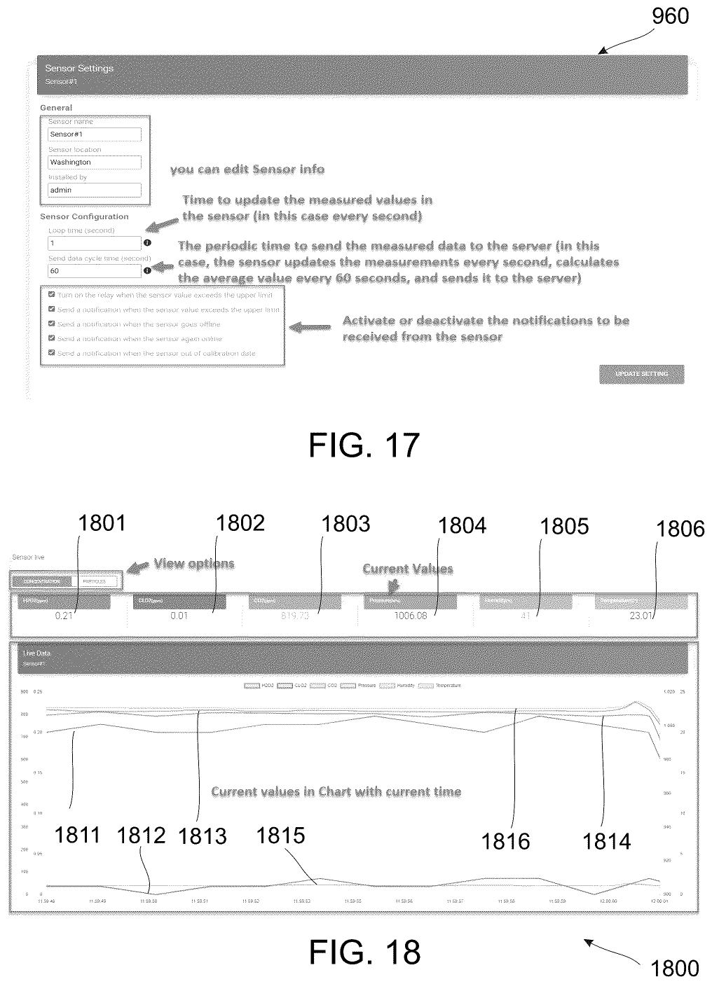

FIG. 17 is a detailed illustration of the settings section 960. The settings section 960 allows a user to enter and manipulate data associated with a respective indoor air quality control system 100, which is in FIG. 17 referred to as a “sensor.”

FIG. 18 shows a live data visualization 1800 of sensor data received from a selected indoor air quality control system 100. The live data view shows a hydrogen peroxide concentration as a numerical H₂O₂ value 1801, measured in ppm, based on a most recently received data packet. Additionally, an H₂O₂ graph 1811 shows a concentration of hydrogen peroxide measured by the indoor air quality control system 100 within the structure 101 over time.

The live data visualization 1800 additionally shows a current ClO₂ concentration 1802, measured in ppm, and ClO₂ graph 1812. The live data visualization 1800 additionally shows a current CO₂ concentration 1803, measured in ppm, and a CO₂ graph 1813. The live data visualization 1800 further shows an air pressure value 1804, measured in hPa, and an air pressure graph 1814. The live data visualization 1800 also shows a relative humidity value 1805, measured in percent, and a humidity graph 1815. The live data visualization 1800 finally shows a relative temperature value 1806, measured in ° C. or ° F., and a temperature graph 1816.

The H₂O₂ concentration, ClO₂ concentration, CO₂ concentration, air pressure, humidity, and temperature are color-coded with one unique color being assigned to each of the displayed air quality factors.

Numerical values are generally displayed in black font. Values that exceed a predetermined threshold are highlighted in a color other than black. For example, the CO₂ concentration 1803 in FIG. 18 is highlighted in orange because its value of 817.24 ppm exceeds a predetermined threshold of 800 ppm.

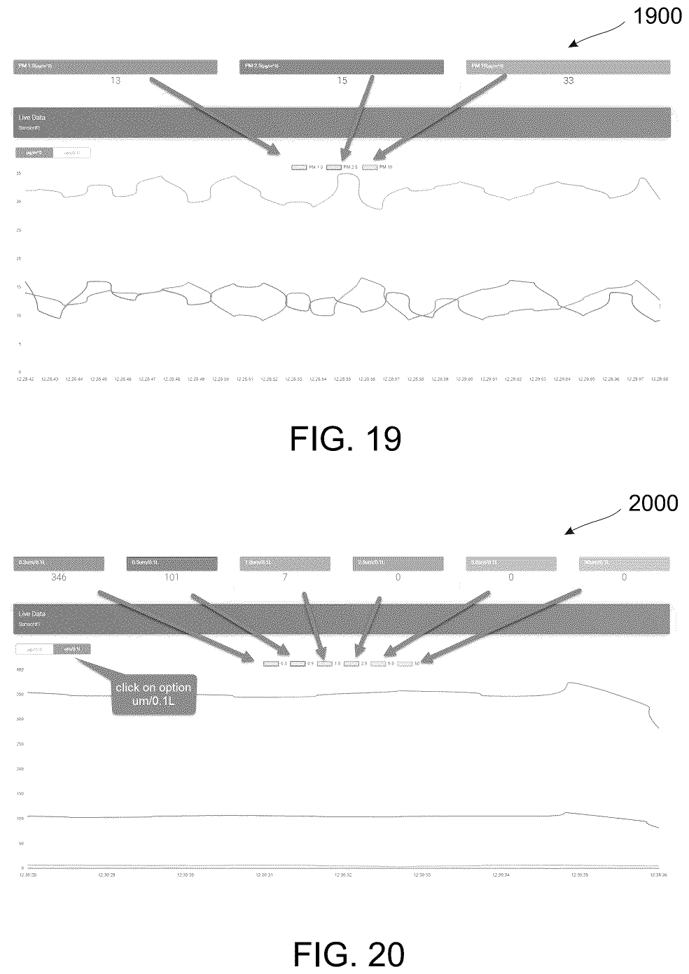

FIG. 19 shows a live particle count data visualization 1900 of further sensor data received from a selected indoor air quality control system 100. The live data visualization 1900 shows three different measurements of particle counts, namely PM 1.0, PM 2.5, and PM10 in μg/m³.

FIG. 20 shows an alternative live particle count data visualization 2000. The graphs are shown in FIG. 20 display six different measurements, namely 0.3, 0.5, 1.0, 2.5, 5.0, 50 um/0.11.

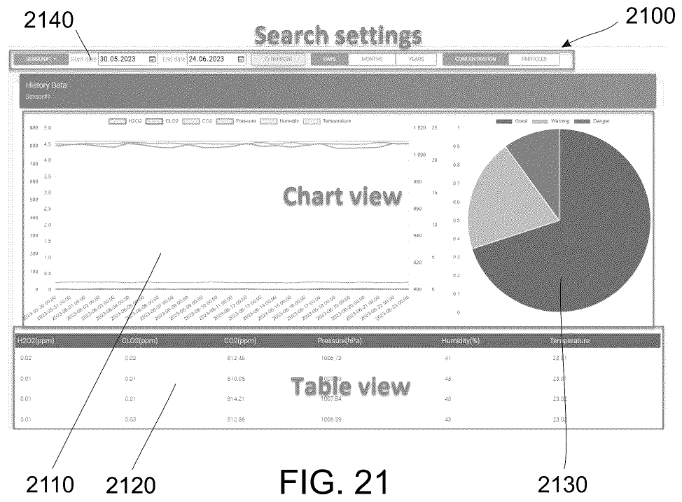

FIG. 21 shows a user interface suitable for reviewing historical air quality data within a selected structure 101. Data is presented in a chart view 2110, a table view 2120, and a histogram view 2130. A search menu 2140 allows filtering data by providing a from-to date range and selecting results to be shown by averaging data received from the indoor air quality control system 100 by day, month, or year.

The histogram view 2130 shows a pie chart in which measurement values below a first threshold are aggregated as “good,” measurement values between the first threshold and a second threshold are aggregated as “warning,” and measurement values above the second threshold are aggregated as “danger” level values. Raw sensor data is displayed in the table view 2120 and can be downloaded, for example in the form of an Excel or CSV table.

In a beneficial application, the structure 101 shown in FIG. 1 may be a greenhouse for growing fruits and vegetables. The structure 101 may alternatively be a storage facility for storing perishable goods, such as fruits and vegetables. The indoor air quality control system 100 may then be used to maintain a controlled atmosphere or dynamically controlled atmosphere in the structure 101. The controlled atmosphere may include a target hydrogen peroxide concentration in the air between 0.5 and 5 ppm. The target hydrogen peroxide concentration is maintained within a closed-loop control. Within the closed loop control, an actual hydrogen peroxide concentration is measured by the sensor 110. The actual hydrogen peroxide concentration is thus a process variable (PV) of the closed-loop control system.

The target hydrogen peroxide concentration is a setpoint (SP) of the closed-loop control system. The hydrogen peroxide nebulizer 121 serves as an actuator of the closed-loop control system. The processor 130 acts as a controller of the closed-loop control system.

The closed-loop control system as shown in FIG. 1 does not have to be connected to the cloud server 140 to function. The indoor air quality control system 100 can operate autonomously if so desired.

Fruits such as apples, pears, and others are seasonal products that often need to be stored for long periods of time to make them available year around. The quality and shelf life of these fruits depend on various factors, including the storage conditions and the composition of the storage atmosphere.

In a controlled atmosphere (CA) storage facility, temperature, humidity, and the composition of the storage atmosphere are monitored and controlled. By reducing the oxygen content and increasing the CO₂ content of air in the storage facility, the natural ripening process is slowed down and the fruit stays fresh for longer. The controlled atmosphere is generally maintained constant over time.

In a dynamically controlled atmosphere (DCA) storage facility, the atmosphere is subject to change based on additional inputs. A DCA storage facility takes into account that stored fruit constantly releases heat, water, carbon dioxide, and ethylene into the ambient air through cellular respiration. In DCA storage, the oxygen content as well as the ethylene and carbon dioxide concentrations are continuously monitored and dynamically controlled. DCA storage facilities generally maintain a lowest possible oxygen value, which is just above the anaerobic reversal point.

Known CA and DCA storage facilities can be further improved by installing the indoor air quality control system 100 as shown in FIG. 1. The indoor air quality control system 100 increases a hydrogen peroxide concentration in the storage facility. Hydrogen peroxide has antimicrobial properties and can help to reduce mold and bacteria. The targeted addition of hydrogen peroxide to the storage atmosphere further improves the shelf life of the fruit, allows fruit to be stored at a higher degree of ripeness, and so contributes to improved quality of the stored fruit.

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations and broad equivalent arrangements that are included within the spirit and scope of the following claims.

What is claimed is:

1. A method for controlling an indoor climate (201, 202, 203), comprising:
sensing, by an air quality control system (100), air quality factors of air within a structure (101);
transmitting sensor data associated with the air quality factors from the air quality control system (100) to a server (140);
analyzing, by the server (140), the sensor data;
calculating a target air quality value by the server (140);
transmitting the target air quality value from the server (140) to the air quality control system (100); and
controlling disbursement of a biocidally active substance within the structure (101),
wherein the air quality factors comprise a sensed concentration of the biocidally active substance, and
wherein controlling disbursement of the biocidally active substance is performed by a closed-loop control with the target air quality value being a setpoint and with the sensed concentration of the biocidally active substance being a process variable.

2. The method as in claim 1, further comprising:
selecting, through a user interface (300, 800), a structure category from the group consisting of a residential building, a hospital, a government office, a school, an office building, a shopping venue, a high-rise building, a theatre, a cinema, an event venue, a hotel, and an ambulance.

3. The method as in claim 2,
wherein calculating the target air quality value by the server (140) is performed in consideration of the structure category.

4. The method as in claim 1, further comprising:
entering, through a user interface (300, 800), a structure volume.

5. The method as in claim 4,
wherein calculating the target air quality value by the server (140) is performed in consideration of the structure volume.

6. The method as in claim 1,
wherein the biocidally active substance is hydrogen peroxide.

7. The method as in claim 1,
wherein the air quality factors further comprise one or more of a ClO₂ concentration, a CO₂ concentration, an ozone concentration, an NO₃ concentration, an air pressure, a relative humidity, and a particle count.

8. The method as in claim 7,
wherein calculating the target air quality value by the server (140) is performed in consideration of one or more of the ClO₂ concentration, the CO₂ concentration, the ozone concentration, the NO₃ concentration, the air pressure, the relative humidity, and the particle count.

9. The method as in claim 1, further comprising:
sensing an occupancy status of the structure (101),
wherein calculating the target air quality value is based on the occupancy status of the structure (101).

10. The method as in claim 1,
wherein controlling disbursement of the biocidally active substance includes generating droplets using an ultrasonic nebulizer.

11. The method as in claim 10,
wherein the droplets have a diameter within a range of 100 nm and 0.5 μm.

12. An indoor air quality control system (100), comprising:
a hydrogen peroxide sensor (111);
a hydrogen peroxide nebulizer (121); and
a processor (130) operatively connected to the hydrogen peroxide sensor (111) and the hydrogen peroxide nebulizer (121),
wherein the processor (130) is configured to control the hydrogen peroxide nebulizer (121) within a closed-loop control system based on an actual concentration of hydrogen peroxide in air surrounding the hydrogen peroxide sensor (111) to maintain a target hydrogen peroxide concentration.

13. The indoor air quality control system (100) as in claim 12,
wherein the target hydrogen peroxide concentration is between 0.1 and 1 ppm.

14. A storage facility for perishable good, comprising:
a structure (101) for storing fruits and vegetables, and
the indoor air quality control system (100) as in claim 12,
wherein the indoor air quality control system (100) maintains a target hydrogen peroxide concentration between 0.1 and 1 ppm.
